(19) United States
(12) Patent Application Publication
(10) Pub. No.: US 2003/0180389 A1
(43) Pub. Date: Sep. 25, 2003

(54) Effervescent glucosamine, chondroitin and MSM formula

(76) Inventor: Cleve Alan Phillips, Hayward, CA (US)

Correspondence Address: WILLIAMS MULLEN, 1 OLD OYSTER POINT ROAD, SUITE 210, NEWPORT NEWS, VA, 23602, US

(21) Appl. No.: 10/394,380

(22) Filed: Mar. 20, 2003

Related U.S. Application Data
(63) Continuation of application No. 09/648,937, filed on Aug. 25, 2000, now abandoned.
(60) Provisional application No. 60/150,552, filed on Aug. 25, 1999.

(51) Int. Cl. A61K 33/04; A61K 9/46; A61K 31/737; A61K 31/7008

(52) U.S. Cl. 424/705; 514/54; 514/62; 424/466

(57) ABSTRACT

A composition which acts to protect, maintain and repair connective tissue in mammals. The composition includes glucosamine, chondroitin sulfate and sulfur in an effervescent base as its major elements. The effervescent base includes one or more acids and one or more bases and may also include a starch, a flavoring agent and a coloring agent. The composition can be formed into a tablet or can be granular. The tablet or granular mixture is dissolved in a neutral pH liquid such as water for consumption purposes.

### CROSS REFERENCE TO RELATED APPLICATION

The present application claims priority from U.S. Provisional Application Ser. No. 60/150,552, filed Aug. 25, 1999.

### BACKGROUND OF THE INVENTION

1. Technical Field of the Invention

The present invention pertains to the field of nutritional formulas. Specifically, the present invention pertains to an improved formula for delivering glucosamine, chondroitin and MSM to the body.

2. Discussion of the Related Art

It is well known that products containing glucosamine, chondroitin and MSM can aid in the protection, maintenance and repair of connective tissue in mammals. Such tissues are regularly exposed to stresses and strains from forces which cause problems such as arthritis, joint inflammation and stiffness. These problems can affect all joints in both humans and animals and can be both painful and debilitating to the sufferer. While the optimal treatment is relief from the force causing the joint problems, this is not always possible.

Other remedies which have been tried for such joint problems are steroids, such as corticosteroids, and other anti-inflammatory materials, such as NSAIDS. High doses of aspirin are also widely used for the treatment of these ailments; Pharmocol. Res. Commun. 10 557-569 (1978) by Vidal et al. In addition, hyaluronic acid and polysulfated glycosaminoglycan are used in veterinary medicine, especially for equines. While these materials often relieve the pain and swelling associated with maladies arising from connective tissue problems, almost all drugs eventually wear out their effectiveness. Furthermore, drugs may also inhibit the body's own natural healing processes, leading to further deterioration of the connective tissue.

Glucosamine provides the joints with the building blocks that are needed to repair damage caused by osteoarthritis or injuries. Specifically, glucosamine provides the raw material needed by the body to manufacture a mucopolysaccharide (called glycosaminoglycan) found in cartilage. Glucosamine sulfate may also play a role in wound healing. Glucosamine is available in several forms. The glucosamine sulfate form (stabilized with a mineral salt) has been the primary form used in the controlled trials of people with osteoarthritis. Glucosamine sulfate is stabilized with one of two mineral salts: sodium chloride (NaCl) or potassium chloride (KCl).

Chondroitin sulfate consists of repeating chains of molecules called mucopolysaccharides. Chondroitin sulfate is a major constituent of cartilage, providing structure, holding water and nutrients, and allowing other molecules to move through cartilage which is an important property, as there is no blood supply to cartilage.

Animal studies indicate that chondroitin sulfate may promote healing of bone, which is consistent with the fact that the majority of glycosaminoglycans found in bone consist of chondroitin sulfate. Chondroitin sulfate also appears to help restore joint function in people with osteoarthritis. Chondroitin and similar compounds are present in the lining of blood vessels and the urinary bladder. They help prevent abnormal movement of blood, urine, or components across the barrier of the vessel or bladder wall. Chondroitin sulfate is classified as a type of glycosaminoglycan; it is rich in sulfur and is related to glucosamine. Glycosaminoglycans affect how the body processes oxalate, a substance linked to kidney stones.

The mineral sulfur is needed for the manufacture of many proteins, including those forming hair, muscles, and skin. Sulfur contributes to fat digestion and absorption, because it is needed to make bile acids. Sulfur is also a constituent of bones, teeth, and collagen (the protein in connective tissue). As a component of insulin, sulfur is needed to regulate blood sugar. Methyl sulfonyl methane (MSM) is one of the key supplements available to provide the body with the extra sulfur it needs to support these processes.

Several previous methods of using glucosamine and/or chondroitin for treating various problems exist. For example, the intravenous administration of glucosamine and derivations thereof are disclosed in U.S. Pat. No. 3,232,836 issued to Carlozzi et al, for assisting in the healing of wounds on the surface of the body. In U.S. Pat. No. 3,682,076 issued to Rovati, the use of glucosamine and salts thereof is disclosed for the treatment of arthritic conditions. Finally, the use of glucosamine salts is also disclosed for the treatment of inflammatory diseases of the gastrointestinal tract in U.S. Pat. No. 4,006,224 issued to Prudden.

Several other methods outline a method in which excess quantities of several of the modified sugars found in the glycosaminoglycans for producing proteoglycans are provided. For example, in U.S. Pat. No. 3,6797,652 issued to Rovati et al, the use of N-acetylglucosamine is disclosed for treating degenerative afflictions of the joints.

Still other methods outline a method in which excess quantities of glycosaminoglycans themselves (with and without various of the modified sugars). For example, in U.S. Pat. No. 3,371,012 issued to Furuhashi, a preservative is disclosed for eye graft material that includes galactose, N-acetylglucosamine (a modified sugar found in the glycosaminoglycans) and chondroitin sulfate (a glycosaminoglycan). Additionally, U.S. Pat. No. 4,486,416 issued to Soll et al, discloses a method of protecting corneal endothelial cells exposed to the trauma of intraocular lens implantation surgery by administering a prophylactically effective amount of chondroitin sulfate. Also, U.S. Pat. No. 5,141,928 issued to Goldman discloses the prevention and treatment of eye injuries using glycosaminoglycan polysulfates.

U.S. Pat. No. 4,983,580 issued to Gibson, discloses methods for enhancing healing of corneal incisions. These methods include the application of a corneal motor composition of fibronectin, chondroitin sulfate and collagen to the incision.

U.S. Pat. No. 4,801,619 issued to Lindblad, the intraarticular administration of hyaluronic acid is disclosed for the treatment of progressive cartilage degeneration caused by proteoglycan degradation.

U.S. Pat. No. 5,364,845 issued to Henderson discloses a method for treating joint problems in humans and animals which comprises administering a composition of glucosamine and chondroitin.

While the above references have, to varying degrees, been useful for their intended purposes, none have proven entirely satisfactory. In particular, the absorption rates of the various compositions disclosed have not been satisfactory.

Thus, it can be seen that there remains a need for a composition which includes glucosamine, chondroitin and MSM in a delivery system which allows for improved absorption of the ingredients.

It is accordingly an object of the present invention to provide a composition for the protection, maintenance and repair of connective tissue in mammals.

It is a further object of the present invention to provide such a composition which is delivered in a manner which allows the compound to carry out its intended effect.

It is a further object of the present invention to provide a composition which contains glucosamine, chondroitin and MSM for protecting, maintaining and repairing connective tissue.

It is a further object of the present invention to provide a composition which exhibits increased absorption rates by means of an effervescent delivery system.

It is yet another object of the present invention to accomplish the foregoing objects in a simple manner.

Additional objects and advantages of the present invention are apparent from the drawings and specification which follow.

### SUMMARY OF THE INVENTION

According to the present invention, the foregoing and additional objects are obtained by providing a composition for protecting, maintaining and repairing connective tissue in mammals which has as its major ingredients glucosamine, chondroitin sulfate and sulfur in an effervescent base. In a preferred embodiment, the glucosamine is glucosamine sulfate and the dose of glucosamine is approximately 1500 mg. The dose of the chondroitin sulfate is preferably approximately 1200 mg. Sulfur is preferably added in the form of methyl sulfonyl methane and the dose ranges from 1000 to 5000 mg but is preferably 1000 mg. The effervescent base includes an acidic ingredient and a basic ingredient. The acidic ingredient can be one of or a mixture of ascorbic acid, citric acid or tartaric acid. The basic ingredient can be one of or a mixture of calcium carbonate, sodium bicarbonate or potassium bicarbonate. The base may further include a starch such as cellulose or alginic acid or other disintegrating starch, a coloring agent and a flavoring agent. The coloring agent may be riboflavin and, if included, the dose of riboflavin is approximately 2.6 mg. The composition is preferably consumed by dissolving either a granular or tablet form of the composition in a neutral pH liquid such as water.

### DETAILED DESCRIPTION OF THE PREFERRED EMBODIMENT

The composition of the present invention acts to protect, maintain and repair connective tissue in mammals. The composition includes glucosamine, chondroitin sulfate and sulfur in an effervescent base as its major elements. The effervescent base includes one or more acids and one or more bases and may also include a starch, a flavoring agent and a coloring agent. The composition can be formed into a tablet or can be granular. The tablet or granular mixture is dissolved in a neutral pH liquid such as water for consumption purposes.

Glucosamine provides the joints with the building blocks that are needed to repair damage caused by osteoarthritis, stress or injuries. Specifically, glucosamine provides the raw material needed by the body to manufacture a mucopolysaccharide (called glycosaminoglycan) found in cartilage. Glucosamine sulfate may also play a role in wound healing. Glucosamine is available in several forms. The glucosamine sulfate form (stabilized with a mineral salt) has been the primary form used in the controlled trials of people with osteoarthritis. Glucosamine sulfate is stabilized with one of two mineral salts: sodium chloride (NaCl) or potassium chloride (KCl).

Glucosamine sulfate does not appear in significant amounts in most diets. Supplemental sources are derived from seashells. While healthy humans do not need to routinely supplement with glucosamine, most research with people who have osteoarthritis has shown that 1500 mg taken daily can be helpful.

Chondroitin sulfate consists of repeating chains of molecules called mucopolysaccharides. Chondroitin sulfate is a major constituent of cartilage, providing structure, holding water and nutrients, and allowing other molecules to move through cartilage which is an important property, as there is no blood supply to cartilage. Animal studies indicate that chondroitin sulfate may promote healing of bone, which is consistent with the fact that the majority of glycosaminoglycans found in bone consist of chondroitin sulfate. Chondroitin sulfate also appears to help restore joint function in people with osteoarthritis. Chondroitin and similar compounds are present in the lining of blood vessels and the urinary bladder. They help prevent abnormal movement of blood, urine, or components across the barrier of the vessel or bladder wall. Chondroitin sulfate is classified as a type of glycosaminoglycan; it is rich in sulfur and is related to glucosamine. Glycosaminoglycans affect how the body processes oxalate, a substance linked to kidney stones. The only significant food source of chondroitin sulfate is animal cartilage. Because the body makes chondroitin, the possibility of a dietary deficiency remains uncertain. Nevertheless, chondroitin sulfate may be reduced in joint cartilage affected by osteoarthritis and possibly other forms of arthritis. For osteoarthritis, a typical level is 1200 mg daily. The ability for chondroitin to be absorbed orally is still under question, which is why it is important to have it immediately available for absorption using the effervescent delivery system.

The mineral sulfur is needed for the manufacture of many proteins, including those forming hair, muscles, and skin. Sulfur contributes to fat digestion and absorption, because it is needed to make bile acids. Sulfur is also a constituent of bones, teeth, and collagen (the protein in connective tissue). As a component of insulin, sulfur is needed to regulate blood sugar. MSM is one of the key supplements available to provide the body with the extra sulfur it needs to support these processes. Most dietary sulfur is consumed as part of certain amino acids in protein-rich foods. MSM is the preferred source for sulfur, but garlic and onion extracts are also good sources. Sulfur in organic forms, such as certain amino acids or in the compound MSM, may be more readily absorbed and used. A dose range of 1000-5000 mg can be taken. Due to the use of the effervescent delivery, the lower range of 1000 mg is easily found effective.

These ingredients are added to an effervescent base and put into tablet or granular form. When dissolved in a neutral pH liquid such as water, this effervescent base provides immediate dissolution of the ingredients and allows them to be immediately available for absorption into the body. The effervescent base is made up of acidic and basic elements which react to release carbon dioxide. These ingredients are described below in more detail.

Ascorbic acid is a water-soluble vitamin (vitamin C) that functions as a powerful antioxidant. It is needed to make collagen, the “glue” that strengthens many parts of the body, such as muscles and blood vessels. Ascorbic acid also plays important roles in wound healing and as a natural antihistamine. Its importance in this product is in its ability to convert MSM into the active sulfur compound utilized by the body. By using effervescence, this highly water-soluble vitamin is delivered into the blood stream with the MSM and supports the body's natural abilities. Vitamin C is the preferred substance, but products such as Broccoli, red peppers, currants, Brussels sprouts, parsley, rose hips, acerola berries, citrus fruit, and strawberries are also considered excellent sources of vitamin C and could be substituted. Other acids which may be included are citric acid or tartaric acid. Generally, any acid which is safe for consumption can be included however, it is preferable to select an acid which tastes good and, if possible, has beneficial side effects.

As for basic ingredients, calcium carbonate, sodium bicarbonate or potassium bicarbonate may be used. These ingredients react with the acidic ingredients to release carbon dioxide. As with the acidic ingredients, any basic ingredient which is safe for consumption can be included, however, it is preferable to select a base which tastes good and, if possible, has beneficial side effects. For example, calcium carbonate provides additional calcium to strengthen bones.

In a preferred embodiment of the present invention, the effervescent base can also include starches, coloring and flavoring. Suitable starches are those that disintegrate such as cellulose or alginic acid. Natural or synthetic flavoring agents can be used. Silica dioxide may also be added to absorb any free water to prevent the ingredients from reacting prematurely.

Riboflavin may be added as the coloring agent. Riboflavin or vitamin B2 is needed to process amino acids and fats, activate vitamin B6 and folic acid, and help convert carbohydrates into ATP, the fuel the body runs on. Under some circumstances, vitamin B2 can act as an antioxidant. Its importance in this formula is to support the natural conversion of the key ingredients into healthy connective tissue. The dose of vitamin B2 included in this composition is 2.6 mg. Due to it being a water-soluble vitamin, it is critical that this vitamin be absorbed as quickly as possible to avoid immediate elimination from the body. Once again, effervescence provides for quick absorption of this element of the composition.

A normal dosage of the present invention for an adult human provides 1500 mg of Glucosamine Sulfate/HCL Complex 40% Hyaluronic Acid (from Glucosamine), 1200 mg of Chondroitin Sulfate, 1000 mg of Methyl Sulfonyl Methane (MSM), 500 mg of Ascorbic Acid/Vitamin C and 2.6 mg of Riboflavin.

### EXAMPLE

A sample composition according to the present invention is set forth below:

| | | | |
|---|---|---|---|
| Fructose | 13 | grams | 61.31% |
| Glucosamine | 1.5 | grams | 7.07% |
| Citric acid | 1.5 | grams | 7.07% |
| Chondroitin | 1.2 | grams | 5.66% |
| Baking soda | 1.0 | grams | 4.72% |
| MSM sulfur | 1.0 | grams | 4.72% |
| Natural Orange Flavor | 0.8 | grams | 3.77% |
| Ascorbic acid | 0.5 | grams | 2.36% |
| Natural Vanilla | 0.3 | grams | 1.41% |
| Silica Dioxide | 0.2 | grams | 0.94% |
| Calcium Carbonate | 0.2 | grams | 0.94% |
| Riboflavin | 0.0026 | grams | 0.01% |
| Sum (grams/dose) | 21.2026 | grams | 100% |

The ingredients in granular form are placed in a sealed, premeasured packet to ensure that the appropriate daily dose is taken. To consume the composition, the ingredients are mixed with water. This causes the effervescent base to react and in turn dissolves the ingredients so that they may be absorbed quickly in the body.

Many improvements, modifications, and additions will be apparent to the skilled artisan without departing from the spirit and scope of the present invention as described herein and defined in the following claims.

What is claimed is:

1. A composition for protecting, maintaining and repairing connective tissue in mammals, comprising quantities of glucosamine, chondroitin sulfate and sulfur in an effervescent base.

2. The composition of claim 1, wherein the glucosamine is glucosamine sulfate.

3. The composition of claim 1, wherein a dose of the glucosamine is approximately 1500 mg.

4. The composition of claim 1, wherein a dose of the chondroitin sulfate is approximately 1200 mg.

5. The composition of claim 1, wherein the sulfur is methyl sulfonyl methane.

6. The composition of claim 1, wherein a dose of the sulfur ranges from 1000 to 5000 mg.

7. The composition of claim 1, wherein a dose of the sulfur is approximately 1000 mg.

8. The composition of claim 1, wherein the effervescent base includes an acidic ingredient and a basic ingredient.

9. The composition of claim 8, wherein the acidic ingredient is ascorbic acid, citric acid or tartaric acid.

10. The composition of claim 8, wherein the acidic ingredient is a mixture of two or more acids selected from the group consisting of ascorbic acid, citric acid and tartaric acid.

11. The composition of claim 8, wherein the basic ingredient is calcium carbonate, sodium bicarbonate or potassium bicarbonate.

12. The composition of claim 8, wherein the basic ingredient is a mixture of two or more bases selected from the group consisting of calcium carbonate, sodium bicarbonate or potassium bicarbonate.

13. The composition of claim 1, wherein the effervescent base further includes a starch.

14. The composition of claim 13, wherein the starch is cellulose or alginic acid.

15. The composition of claim 1, wherein the effervescent base further includes a flavoring agent.

16. The composition of claim 1, wherein the effervescent base further includes a coloring agent.

17. The composition of claim 16, wherein the coloring agent comprises riboflavin.

18. The composition of claim 17, wherein a dose of the riboflavin is approximately 2.6 mg.

19. The composition of claim 1, wherein the glucosamine, chondroitin sulfate, and sulfur in an effervescent base are dissolved in a neutral pH liquid.

20. The composition of claim 19, wherein the glucosamine, chondroitin sulfate and sulfur in an effervescent base are dissolved in water.
